SEQUENCE LISTING (2 sequences)

SEQ ID NO: 1 - protein, 25 aa, mouse
  ref <301>: AUTHORS: Y. Zhao et al.
  ref <302>: TITLE: Enhancing tumor targeting and apoptosis using noncovalent antibody homodimers
  ref <303>: JOURNAL: J. Immunother.
  ref <304>: VOLUME: 25
  ref <305>: ISSUE: 5
  ref <306>: PAGES: 396-404
  ref <307>: DATE: 2002-09
ASRNKANDYTTDYSASVKGRFIVSR

SEQ ID NO: 2 - protein, 17 aa, mouse
KGEGAAVLLPVLLAAPG

(19) United States
(12) Patent Application Publication
(10) Pub. No.: US 2004/0185039 A1
(43) Pub. Date: Sep. 23, 2004

(54) Therapeutic applications of noncovalent dimerizing antibodies

(76) Inventors: Heinz Kohler, Lexington, KY (US); Alton C. Morgan JR., Arlington, WA (US)

Correspondence Address: JAMES H. MEADOWS AND MEDICUS ASSOCIATES, 2804 KENTUCKY, JOPLIN, MO, 64804, US

(21) Appl. No.: 10/652,864

(22) Filed: Aug. 29, 2003

Related U.S. Application Data
(60) Provisional application No. 60/407,421, filed on Aug. 30, 2002.

(51) Int. Cl. A61K 39/395

(52) U.S. Cl. 424/131.1

(57) ABSTRACT

Compositions and methods for providing antibodies having noncovalent, self-binding properties are disclosed. Such autophilic antibodies can bind cellular receptors to promote apoptosis of target cells and enhance therapeutic efficacies in the treatment of patients with debilitating or life-threatening diseases. Representative diseases targeted by the autophilic antibodies are lymphomas, breast cancers, colon cancers, and melanomas. Autoimmune disorders, Alzheimer's disease, and other neuro-degenerative conditions, as well as graft or transplant rejection, are among other treatable conditions.

### REFERENCE TO RELATED APPLICATION

This application claims the benefit of U.S. Provisional Application 60/407,421, filed Aug. 30, 2002.

### BACKGROUND OF THE INVENTION

The present invention relates to antibody formulations and methods of administration in the treatment of a variety of diseases, especially those treatable with passive antibody therapy.

Antibodies have emerged as a major therapeutic tool for the treatment of chronic diseases such as cancer and autoimmune disorders. The principal advantage of these biological agents lies in the unique targeting of disease-causing cells or molecules, which can spare healthy tissue and normal products of the body. However, antibodies that exhibit ideal specificities often fail in pre-clinical and clinical evaluations because of inefficient targeting and/or low biological activity.

It is known that a major mechanism by which therapeutic antibodies attack cancer cells is through the induction of apoptosis. Apoptosis is triggered by crosslinking cellular receptors that are part of the apoptosis signal pathway. For example, crosslinking the CD20 cellular receptor on B-cells delivers a strong apoptosis signal in malignant lymphomas (Zhao Y., et al., 2002). In a similar manner, crosslinking the B-cell antigen receptor by means of antibodies also induces apoptosis in B-cell tumors (Ghetie M., et al., 1997). Crosslinking of cellular receptors also increases the avidity of binding of antibody to its target antigen, and thus is likely to increase all cell surface-dependent therapeutic mechanisms, such as complement-mediated killing and complement-dependent opsonization and phagocytosis, antibody-dependent cellular cytotoxicity (ADCC), as well as enhanced inhibition of cell growth or alterations in metabolic pathways within cells through increased binding to and blockade of cellular receptors when using antibodies targeted to cellular receptors.

It was recently demonstrated (Zhao Y., et al., 2002) that antibodies capable of forming dimers and polymers, without being crosslinked by covalent means prior to targeting, enhance apoptosis over that induced by non-dimerizing antibodies. These non-covalent, dimerizing antibodies are formed by attaching a peptide group, which induces dimerization or multimerization only after the modified antibody attaches to its cell surface target. This phenomenon of “differential oligomerization” can also be demonstrated by immobilizing a portion of modified antibody to a plastic surface and subsequently demonstrating binding of modified antibody (with peptide). In contrast, this type of modified antibody, also termed an “autophilic” antibody, forms an equilibrium in solution between monomeric and dimeric forms heavily favored towards the monomeric state (Kaveri S., et al., 1990).

U.S. Pat. No. 5,800,991 (issued to Haley et al.) discloses a method for immunodetection of an antigen that employs a labeled antibody wherein the labeled antibody is a conjugate with a nucleotide photoaffinity compound. U.S. Pat. No. 6,238,667 (issued to Kohler) discloses a method of chemically cross-linking a peptide to an affinity site on antibodies. One aspect of the method entails photochemically activating a peptide containing an azido group, and reacting the activated peptide with an antibody. The affinity site of the antibody is highly conserved and consists of framework residues within the variable domain domains of the heavy and light chains of the antibody. The site of cross-linking is located away from the antigen-binding site in the Fv domain, thereby avoiding compromise of antigen recognition. Moreover, U.S. patent Pub. No. 2003/0103984 (Kohler) discloses a fusion protein comprising antibody and peptide domains in which the peptide domain can have autophilic activity.

Others have proposed the use of hybrid molecules for therapeutic purposes wherein the hybrid molecules comprise two distinct domains covalently linked. For instance, U.S. Pat. No. 6,482,586 (issued to Arab et al.) proposes covalent hybrid compositions for use in intracellular targeting. U.S. Pat. No. 6,406,693 (issued to Thorpe et al.) proposes antibodies and conjugates for killing tumor vascular endothelial cells by binding to aminophospholipid on the luminal surface.

These are but a few of the approaches that have been used to enhance therapeutic efficacy of monoclonal antibodies that in their native or “humanized” state, are not effective in killing their targets or triggering a biological function affording therapeutic efficacy. In contrast to and in addition to these approaches, autophilic antibodies alone can self-associate to enhance apoptosis or together with these other approaches enhance their therapeutic effects.

The effects of autophilic non-covalent antibodies have been clearly demonstrated in vitro using different target tumors and antibodies, the potential to enhance apoptosis remains to be evaluated in vivo. Their ability to lead to enhanced therapeutic effects in animal models has also been demonstrated with rare, naturally-occurring autophilic antibodies (Kang, C-Y. et al., 1986). Superior efficacy in such models depends on antibody effector functions such as complement-mediated killing, opsonization and phagocytosis. The enhancement of other therapeutic mechanisms, such as use of immunoconjugates, remains to be demonstrated in vivo.

A continuing need exists for new therapies in the treatment of cancer, autoimmune disorders, and graft rejection. It is believed that autophilic, non-covalent antibody dimers and polymers offer great potential for the treatment of human malignancies and other metabolic and immunological disorders.

### SUMMARY OF THE INVENTION

The present invention is directed to autophilic antibody compositions and methods of obtaining enhanced therapeutic efficacies in the treatment of patients with debilitating or life-threatening diseases. Among the subject diseases are those treatable with passive antibodies, such as cancers derived from lymphatic, epithelial or endothelial cells. Autoimmune disorders, Alzheimer's disease, and other neuro-degenerative conditions, as well as artifacts of a functioning immune system such as graft or transplant rejection, are also among the treatable conditions. Antibodies according to the present invention have the unusual property of spontaneously binding to self only after first binding to their target antigen (differential oligomerization).

An autophilic antibody of the invention is preferably formed by one of several methods, including by chemically crosslinking a peptide capable of self-binding to an antibody's Fc region through oxidation of an N-linked carbohydrate. Alternatively, the autophilic peptide can be linked to antibody through the nucleotide or tryptophan binding site or in less specific methods, such as through antibody epsilon amino groups or sulfhydryl groups obtained through partial reduction of the antibody. The preferred methods use crosslinking to groups within the antibody molecule not involved directly in antigen binding.

In a preferred embodiment, the antibody is a monoclonal antibody (Mab) specific for a B-cell receptor (BCR) of a murine or human B-cell tumor. Such autophilic antibodies can bind to their respective tumor target cells with increased efficiency as determined by fluorescence-activated cell sorting (FACS) analysis. They also can induce greater apoptosis of target tumor cells than control antibodies. Autophilically-modified antibodies are observed to inhibit tumor growth in culture more efficiently than control antibodies and provide stronger protection against bacterial infection than non-self-binding antibodies having identical specificity and affinity.

The present invention affords antibodies having self-binding properties that mimic those of rare, naturally occurring, autophilic antibodies. The invention thereby offers a simple and attractive alternative to covalent dimerization and other engineering approaches directed to enhancing the therapeutic potential of antibodies.

### DESCRIPTION OF THE INVENTION

The present invention relates to non-covalent, dimerizing antibodies having enhanced therapeutic potencies. Such antibodies are referred as “autophilic” antibodies and exist in nature (Kang C-Y. et al., 1986) or can be produced by chemical and genetic manipulations. Autophilic antibodies belong to the class of superantibodies—antibodies that exhibit one or more properties not usually associated with antibodies (Kohler H., et al., 1998; Kohler H., 2000). The defined class of superantibodies comprises catalytic, membrane-penetrating, and autophilic antibodies and includes many antibodies exhibiting superior targeting and therapeutic properties.

In a preferred aspect of the invention, a patient who suffers from a debilitating or potentially life-threatening disease or condition is administered at least one subject autophilic antibody in an amount effective to alleviate symptoms of the disease or condition. A disease or condition contemplated for treatment by an antibody of the invention can be a malignancy, neoplasm, cancer, auto-immune disorder, Alzheimer's disease or other neuro-degenerative condition, or graft or transplantation rejection.

According to the principles of the present invention, an autophilic antibody is preferably administered in one or more dosage amounts substantially identical to or less than those practicable for unmodified antibodies. Thus, in the treatment of a lymphoma or a breast cancer, an autophilic antibody of the invention is administered in one or more dose amounts substantially identical to that used for RITUXAN or HERCEPTIN. For example, treatment with HERCEPTIN (a humanized monoclonal anti-HER2/neu antibody) employs an antibody concentration of about 10 mg/ml. Intravenous infusion over 90 minutes provides a total dose of 250 mg on day 0. Beginning at day 7, 100 mg is administered weekly for a total of 10 doses. The dosing regimen is reduced gradually from 250 mg to 100 mg to a maintenance dose of 50 mg. Similar dosage regimens to that for HERCEPTIN can be employed with autophilic antibodies, with any adjustments being well within the capabilities of a skilled practitioner.

In another aspect of the invention, a method of potentiating apoptosis of targeted cells of a patient comprises administering a first autophilic antibody-peptide conjugate and a second antibody that recognizes the peptide domain of the conjugate. In this embodiment, the antibody-peptide conjugate recognizes the extracellular region of a transmembrane receptor of the target cell. Owing to its homodimerization property, the antibody-peptide conjugate can bind more avidly to the target than the corresponding antibody lacking the self-binding peptide domain. Moreover, whenever the autophilic antibodies bind to two or more receptors, with those receptors being brought in close proximity due to the self-binding property of the antibodies, an apoptosis signal within the cell can be triggered. In those instances when the peptide domain of the conjugate presents an exposed epitope, a second antibody, specific for the autophilic peptide, can be administered, bind to the modified antibody, and enhance the process of crosslinking and even cause temporary clearance of the target antigen. If the target antigen is a receptor, clearance from the cell surface, endocytosis, and degradation will subsequently require synthesis of new receptor protein, meaning that the biological function of the receptor will be more effectively inhibited for a longer period than using either a simple blocking antibody or small molecule inhibitor. Alternatively, the second antibody can bear a radiolabel or other potentially therapeutic substance, so that when administered it can attack the targeted cells. The key to use of this second antibody is that antibody's specificity. The autophilic peptide, though naturally occurring, is present on only a small number of murine imunoglobulins. Thus, antibody specific to this peptide will have the requisite selectivity to be used in vivo.

The present invention also contemplates a method of producing therapeutic autophilic antibodies. The antibodies can be produced by chemical or genetic engineering techniques. For instance, a peptide component of an autophilic antibody can be attached to the immunoglobulin component via its variable domain structures using azido-tryptophan or azido-purine photoactivation crosslinking. In this approach, the peptide attaches to the variable domain at a location that does not interfere with antigen recognition. This method can incorporate two peptide moieties into a single immunoglobulin molecule. See, e.g., U.S. Pat. No. 6,238,667, U.S. Reissued Patent RE38,008, U.S. Pat. No. 5,635,180, and U.S. Pat. No. 5,106,951, the disclosures of which are incorporated herein by reference.

In a preferred aspect, an autophilic antibody contains a self-binding peptide component, such as the autophilic T15 peptide, which comprises regions of CDR2 and FR3 of the murine germline-encoded S107/TEPC15 (T15) antibody. The T15 peptide has amino acid sequence: ASRNKANDYTTDYSASVKGRFIVSR (SEQ ID NO.: 1) (Kang CY, et al., 1988). Its self-binding property has been shown to be antigen-independent, thereby suggesting attachment of the peptide to monomeric antibodies can impart autophilic and increased avidity properties to the antibodies (Kaveri S., et al., 1991). The T15 peptide can be photo-crosslinked to a heterocycle or nucleotide affinity site of the immunoglobulin to produce the autophilic antibody. Alternatively, the T15 peptide can be crosslinked to a carbohydrate site of the Fc portion or to an amino or sulfhydryl group of the immunoglobulin. Also, the autophilic antibody can be conveniently expressed as a fusion protein of the T15 peptide and whole immunoglobulin, or fragment thereof.

The homodimerizing antibodies of the present invention preferably bond non-covalently with other such conjugated antibodies when bound to their target antigen(s), usually a cell-surface, trans-membrane receptor(s). However, premature formation of dimers or multimers of the antibodies may lead to difficulties in manufacturing, such as during purification and concentration, as well as drawbacks in administration, such as in complement fixation, which may lead to avoidable side effects. As such, the autophilic antibody-peptide conjugates should be formulated to reduce this dimerizing potential and maximize monomericity while in solution and before administration. It has been found that solution dimerization can be reduced or mitigated by formulating the composition with salt concentrations of 0.5M or more, low levels of SDS or other various detergents such as those of an anionic nature, or by modifications of the antibody to decrease its isoelectric point as with succinyl anhydride.

An assay method is also contemplated that permits pre-selection of target antigens most suitable as targets for the autophilic antibodies of the present invention. Such method entails the in vitro assay of apoptosis with multiple antigen-positive target cell lines, and if possible, fresh isolates of antigen-positive cells. The assay may be modified to include a source of complement and or effector cells including non-isolated or isolated fractions of peripheral blood cells, lymph node, thoracic duct or spleen cells. Cells may be enumerated by pre-labeling, such as with ⁵¹Cr or ¹³¹I-UDR, or by counting with FACS. Positive results in this assay predict a positive outcome using an autophilic conjugate. However, negative results in the assay do not mean that subsequent conjugation with autophilic peptide will not improve one or more antibody effector properties.

Autophilic antibodies of the present invention have a higher potential for forming dimers when conjugated to suitable peptides and can have a higher therapeutic potency through triggering apoptosis. Suitable animal models for testing efficacy of the aforementioned autophilic antibodies include severely compromised immunodeficient (SCID) mice or nude mice bearing human tumor xenografts.

A method of enhancing apoptosis, complement fixation, or effector cell-mediated killing of targets is also disclosed employing an autophilic conjugate of the invention. Allowing time for binding to the target cell and clearance from normal tissues, a second anti-autophilic peptide antibody is administered. Whenever a non-native peptide, e.g, the T15 sequence, is employed as the peptide moiety, an anti-T15 peptide antibody only recognizes and binds to antibodies conjugated with the sequence.

A further method of enhancing apoptosis, complement fixation, or effector cell-mediated killing of targets is contemplated, which employs an autophilic conjugate of the invention in which a template peptide, e.g., T15, has been modified to enhance the crosslinking potential of the autophilic antibodies. Such functionally enhanced peptides are determined by producing a series of synthetic peptides with conservative substitutions at each amino acid position within the template sequence and then testing this library of peptides for self-binding or for binding to the original sequence. Those peptides with superior binding to the original sequence are then conjugated to immunoglobulins and the resultant conjugates are tested for potency.

Autophilic antibody conjugates bearing a combination of bioactive peptides are also contemplated. An example is an antibody conjugate that bears a T15 peptide conjugated to the carbohydrate of an antibody and an MTS membrane translocation peptide (Y. Zhao et al., 2003; Y. Lin et al., 1995) having amino acid sequence KGEGAAVLLPVLLAAPG (SEQ ID NO. 2) conjugated to the tryptophan-binding site. The T15 peptide affords autophilicity to the conjugate and the MTS sequence affords the ability to penetrate into cells. Such a conjugate can target cancer cells for radio-immunotherapy when its antibody region targets a primarily intracellular, tumor-associated antigen, such as carcino-embryonic antigen (CEA) (See, e.g., U.S. Pat. No. 6,238,667). The autophilic conjugate, upon administration, targets CEA-bearing, colon carcinoma cells, is internalized by translocation of the antibody mediated by the MTS peptide, and is enabled to bind to the more prevalent intracellular form of CEA. Crosslinking of CEA antibody with, for instance, a therapeutic isotope such as ¹³¹I will be retained in a cell longer than unmodified, labeled antibody and will deliver a higher radioactive dose to the tumor. In addition, such therapeutic isotopes as ¹²⁵I, which release beta particles of short path length and are not normally considered useful for therapy, can, when delivered intracellularly in closer proximity to the nucleus, be efficacious against certain targets, especially those of lymphoid origin and accessible in the blood and lymph tissues.

The following examples are presented to illustrate certain aspects of the invention, but do not limit it.

### EXAMPLES

### Example 1

### Crosslinking of T15 peptide to Two Mabs Specific for B-Cell Receptor

Cell Line and Antibodies. The human B-cell tumor line (Su-DHL4) and murine B-cell tumor line (38C13) are grown in RPMI 1640 medium (supplemented with 10% fetal bovine serum, 2 μmol/L glutamine, 10 μmol/L HEPES, 50 U/mL penicillin, and 50 μg/mL streptomycin, 50 μmol/L 2-mercaptoethanol) at 37° C. under 5% carbon dioxide. Two mAb 5D10 and SIC5, specific for the human or murine BCR, respectively, were used in this study. The antibodies are purified from the culture supernatant by protein G and protein A affinity chromatography.

Synthesis of Antibody-Peptide Conjugate. T15H peptide (ASRNKANDYTTDYSASVKGRFIVSR), a VH-derived peptide from a self-binding antibody-T15, was synthesized by Genemed Synthesis (San Francisco, Calif., U.S.A.). Antibodies were dialyzed against PBS (pH 6.0) and 1/10 volume of 200 μmol/L sodium periodate was added and incubated at 4° C. for 30 minutes in the dark. The reaction was stopped by adding glycerol to 30 μmol/L, and the sample was dialyzed at 4° C. for 30 minutes against PBS (pH 7.0). One hundred times molecular excess of T15H or scrambled peptide was added to the antibodies and incubated at 37° C. for 1 hour. L-Lysine was added and incubated at 37° C. for 30 minutes to block the remained aldehyde group. The same oxidation reaction steps (except adding the peptides) were applied to antibodies used as controls. After the blocking step, the antibody conjugates were dialyzed against PBS (pH 7.2) overnight.

Ig Capture ELISA. Four μg/mL of S1C5-T15H was coated to Costar vinyl assay plates (Costar, Cambridge, Mass.). After blocking with 3% BSA solution, 8 μg/mL of photobiotinylated S1C5-T15H, S1C5-scrambled peptide conjugate, and control S1C5 were added to the first wells, and 1:1 dilution was performed. The antibodies were incubated for 2 hours at room temperature. After washing with PBS buffer, Avidin-HRP (Sigma, St. Louis, Mo.) was added as a 1:2500 dilution. The binding antibodies were visualized by adding substrate o-phenylenediamine.

Size Exclusion Chromatography. Antibody conjugate was chromatographed on a 75 mL Sephacryl 300 HR column (Pharmacia, Peapack, N.J.). 1:10 diluted PBS (pH 7.2) was chosen as elution buffer. Fractions (0.5 mL/each) were collected and aliquots (100 μL) were assayed on antihuman IgG capture ELISA. The ELISA reading (OD 490 nm) is 10 plotted against elution volume.

Viability Assay for Antibody-Treated Cells. The lymphoma cells were grown in 96-well tissue culture wells in 1-mL medium. 2 μg of antibodies or antibody-peptide conjugates were added and incubated for various times as described herein. Ten μL aliquots from the cell suspension were used to determine viability by using trypan blue exclusion.

FACS Assay of the B-Cell Lymphoma. The Su-DHL4 and 38C13 cells were fixed with 1% paraformaldehyde. 1×10⁶ cells were suspended in 50 μL of staining buffer (Hank's balanced salt solution, containing 0.1% NaN3, 1.0% BSA), then 1.5 μg of photobiotinylated SIC5-T15H conjugates was added and incubated for 30 minutes on ice. Control antibodies and antibody-scrambled T15 peptide conjugates served as controls. The cells were washed twice with staining buffer before Avidin-FITC (Sigma) was added to the cells for 30 minutes on ice. Then the cells were washed twice with staining buffer, re-suspended in 200 μL PBS and analyzed by flow cytometry.

Hoechst-Merocyanin 540 Staining to Detect Apoptosis. 1×10⁶ of lymphoma cells were placed into 24-well tissue culture wells. Four μg of antibodies or antibody-peptide conjugates were added and incubated for various times as described herein. 1×10⁶ cells were removed from the culture, re-suspended in 900 μL cold PBS (pH 7.2). One hundred μL of Hoechst 33342 (50 μg/mL; Molecular Probe, Eugene, Oreg., U.S.A.) was added, the cells were incubated at 37° C. for 30 minutes in the dark. The cells were centrifuged and re-suspended in 100 μL PBS. Then, 4 μL of MC540 solution (Molecular Probe) was added, and a 20-minute incubation was performed at room temperature in the dark. The cells were pelleted, re-suspended in 1 mL cold PBS (pH 7.2), and analyzed by flow cytometry.

Results

Characterization of Autophilic Antibodies. The T15H (24-mer) peptide was crosslinked to two murine mAb (SIC5 and 5D10), using carbohydrate periodate conjugation. The mAb S1C5 (IgG1) is specific for the tumor idiotype of the mouse 38C13 B-cell line and the 5D10 antibody for the human Su-DHL4 B-cell tumor. Both antibodies recognize unique idiotypes of the BCR IgM on the B-cell tumors.

Self-Binding Behavior can Easily be Demonstrated by ELISA. The autophilic self-binding effect was studied with the T15H peptide-crosslinked mAb SIC15. The T15H-crosslinked S1C5 binds to insolubilized S1C5-T15H detected by biotin-avidin ELISA. Control S1C5 does not bind significantly to S1C5-T15H or S1C5 crosslinked with a scrambled peptide. Similar self-binding of T15H peptide-crosslinked mAb 5D10 to insolubilized T15H-5D10 was also observed. The specificity of the peptide mediated autophilic effect was tested using the 24-mer peptide T15H itself as an inhibitor. Only the T15H peptide inhibited S1C5-T15H and 5D10- T15H self-binding while the control-scrambled peptide did not inhibit it. These results are similar to the previously published inhibition data with the naturally occurring autophilic T15/S107 antibody.

T15H-Antibody Conjugates Form an Equilibrium of Monomer and Dimer in Solution. The noncovalent nature of the self-aggregation of T15H-linked antibodies raises the question of its physical state in solution. To address this issue, we analyzed the molecular species of T15H-linked mAb using gel electrophoresis and sizing gel filtration. The electrophoretic mobility of control and T15H peptide conjugated S1C5 and 5D10 under reducing and nonreducing conditions show no differences, indicating the absence of chemical bonds between the antibody chains. The molecular species of the peptide-conjugated antibodies (5D10-T15H) was further analyzed by size exclusion chromatography. The elution profile indicated two immunoglobulin species of different size. The larger first peak eluted in the position of an antibody dimer. The second smaller peak eluted in the position of nonconjugated 5D10 antibody. The appearance of two peaks resembled monomer and dimer antibodies and could indicate that either a fraction of antibodies was not modified to polymerize, or that the modification was complete and the antibody establishes an equilibrium of dimers and monomers. To test the latter possibility, material from both peaks were subjected to a second gel filtration on the same column. Reruns of both peaks yielded again two peaks at the same position as in the first chromatography. These data show that the T15H peptide-linked antibodies exist in solution as two distinct molecular species in equilibrium as monomer and dimer. Enhanced Binding of Autophilic Antibodies to Tumors. The binding of the peptide-conjugated antibodies against their respective tumor targets was compared with that of the control antibodies in indirect fluorescence activated cell sorting (FACS). As control, antibodies linked with a scrambled peptide were included. The fluorescence intensity of the T15H-S1C5 on 38C13 cells is compared with that by the control S1C5 and the scrambled peptide S1C5. The difference in mean fluorescence channels between S1C5-T15H and controls was greater than 10-fold. Similarly, the FACS analysis of autophilic 5D10-T15H on Su-DHL4 cells shows enhancement of binding over binding of control 5D10 and control peptide-crosslinked 5D10. In both tumor systems, the conjugation of the T15H peptide to tumor-specific antibody enhanced the FACS signals over control antibodies used at the same concentration. The enhancement of fluorescence can be explained with the increase of targeting antibodies caused by self-aggregation and lattice formation on the surface of the tumor cells.

Inhibition of Tumor Growth. Antibodies binding to the BCR induce crosslinking of the BCR, which, in turn, inhibits cell proliferation and produces a death signal. Furthermore, chemically dimerized antibodies directed against a B-cell tumor induce hyper-crosslinking of the BCR followed by inhibition of cell division and apoptosis of the tumor. To see if similar enhancement of the antitumor effects of dimerizing antibody were induced by our noncovalent, dimerizing T15H-linked antibodies, the two B cell tumors were cultured in the absence or presence of control and T15H-linked antibodies. Co-culture of both tumors, 38C13 and Su-DHL4, with their respective T15H-linked antibodies inhibited the cell growth significantly better compared with the control antibodies. To test the tumor target specificity of autophilic antibodies in growth inhibition, criss-cross experiments were performed with the 38C13 and Su-DHL-4 cell lines. Inhibition of 38C13 cell growth with S1C5-T15H was statistically greater than mismatched 5D10-T15H. Similar results on the specificity of autophilic antibodies were obtained with the Su-DHL4 cells.

Induction of Apoptosis. As suggested by earlier studies, the antitumor effect of antibodies directed against the BCR of B-cell lymphomas in vitro and in vivo might be caused by the induction of apoptosis. Aliquots of tumor cells (38C13 and Su-DHL-4) cultured in the presence of control or T15H-linked antibodies were analyzed for apoptosis using a double stain FACS protocol. 38C13 and Su-DHL4 cells underwent a moderate amount of apoptosis without antibodies over a 6, respectively 18-hour culture. This apoptosis was enhanced when the respective antibody was added. However, when the T15H-linked antibodies were added, the accumulated number of apoptotic 38C13 cells was almost doubled, and apoptosis of Su-DHL4 cells was more than doubled during the entire culture.

Discussion

The biologic advantage of the autophilic property is exemplified with the S107/T15 anti-phosphorylcholine antibody. This self-binding antibody is several times more potent in protecting immune-deficient mice against infection with pneumococci pneumoniae than nonself-binding antibodies with the same antigen specificity and affinity.

As shown here, the autophilic antibody function can be transferred to other antibodies by chemically crosslinking a peptide derived from the T15 VH germline sequence. The modified antibody mimics the self-binding property of the T15/S107 antibody, producing a dimeric antibody with increased avidity and enhanced targeting. This approach is an attractive alternative to strategies of improving the targeting of antibodies by either chemical crosslinking or by antibody engineering. Enhancing the binding of autophilic engineered antibodies to the BCR of B-cell tumor increases the strength of the death signals leading to profound inhibition of cell proliferation in culture. Even though the doubling of apoptosis is demonstrated here, it appears that other mechanisms of growth inhibition are involved.

Crosslinking the BCR of the mature murine B-cell lymphoma A20 can protect against CD95 mediated apoptosis. This anti-apoptotic activity of engagement of the BCR by crosslinking antibodies is highly restricted to the time window of CD95 stimulation and is not dependent upon protein synthesis. The finding that BCR hypercrosslinking per se is pro-apoptotic is not at variance with reports on the anti-apoptotic activity of the BCR engagement, because it can be a result of the use of less mature B-cell lines in our study, to different strength of delivered signals by homodimerizing antibodies, or to Fas-independent apoptosis.

The use of two BCR idiotope-specific antibodies against different tumors offered the opportunity to test the biologic effect of targeting receptors other then the idiotope specific BCR. In criss-cross experiments with autophilic antibodies binding in FACS analysis and inhibition of growth in vitro show a significant enhancement only with the autophilic matched antibody. In this context, it is interesting to speculate whether enhanced tumor targeting would also augment cellular effector functions. Such in vitro and in vivo experiments are in progress.

In an earlier study using chemically homodimerized antibodies, the Fc domain was not involved in the augmentation of growth inhibition and tumor cells lacking Fc receptors were susceptible to the antigrowth activity of homodimers. Thus, the antitumor effect induced by dimerizing antibodies would not be restricted to tumors expressing Fc-receptors.

The described approach of transferring the naturally occurring autophilic property to other antibodies thereby enhancing their antitumor effect outlines a general method to improve the therapeutic efficacy of antibodies in passive immunotherapy. Such noncovalent antibody complexes offer several advantages over chemically crosslinked antibodies: (i) the equilibrium between monomer and noncovalent homopolymers prevents the formation of precipitating nonphysiologic complexes in solution; (ii) autophilic conversion does not compromise the structural integrity of antibodies; and (iii) the method is simple and efficient and does not require a purification step typically needed for chemically crosslinked homodimers that reduces the yield of active Ig dimers. One possible limitation of the approach of using dimerizing antibodies might be the ability to penetrate a large tumor mass. Because the homophilic peptide is of murine origin, it might be immunogenic in humans. Thus, it could be necessary to humanize the murine peptide based on sequence and structural homology using computer modeling. The demonstration that adding a single peptide to the structure of antibodies increases the amount of antibody bound to targets and the antitumor activity encourages attempts to engineer recombinant antibodies expressing the autophilic activity.

### Example 2

### MTS-Conujugated Antibody Facilitates Internalization

Cell line and antibodies. Human Jurkat T cells were grown in RPMI 1640 supplemented with 10% fetal bovine serum and antibiotic (penicillin, streptomycin and amphotericin). Rabbit polyclonal anti-active caspase-3 antibody (#9661S) and anti cleaved-fodrin, i.e. alpha II spectrins (#2121S) were purchased from Cell Signaling, Inc (Beverly, Mass.). Monoclonal (rabbit) anti-active caspase-3 antibody (#C92-605) was purchased from BD PharMingen (San Diego, Calif.). Mouse monoclonal antibody 3H1 (anti-CEA) was purified from cell-culture supernatant by protein G affinity chromatography. Anti-mouse and anti-rabbit HRP-conjugated secondary antibodies were purchased from Santa Cruz Biotechnologies, Inc. ApoAlert Caspase-3 Fluorescent Assay kit was purchased from Clonetech Laboratories (Palo Alto, Calif.). The Cell Death Detection ELISA was purchased from Roche Applied Science (Indianapolis, Ind.).

Synthesis of MTS peptide-antibody conjugate. MTS peptide (KGEGAAVLLPVLLAAPG) is a signal peptide-based membrane translocation sequence and was synthesized by Genemed Synthesis (San Francisco, Calif.). Antibodies were dialyzed against PBS (pH6.0) buffer, oxidized by adding 1/10 volume of 200 mmol/L sodium periodate and incubating at 4° C. for 30 min in the dark. Adding glycerol to a final concentration of 30 mM terminated the oxidation step. Samples were subsequently dialyzed at 4° C. for 1 h against 1×PBS (pH6.0) buffer. The MTS peptide (50 times molar excess) was added to couple the antibodies and the samples were incubated at 37° C. for 1 h and the resulting antibody-peptide conjugate was dialyzed against 1×PBS (pH 7.4).

Effect of MTS-conjugated antibody on cell growth. Jurkat cells (2.5×10⁵) were seeded into 96-well culture plate. After incubation with 0.5 μg MTS-antibody conjugates for 6, 12, 18 and 24 hour, aliquots were removed and viability was determined by trypan blue exclusion.

Study of antibody internalization by ELISA. Jurkat cells, grown in 1-ml medium in a 6-well culture plate, were incubated with 2 μg of unconjugated or MTS conjugated antibodies for 0, 1, 3, 6, 12 and 18 h. The cells were centrifuged and the culture supernatant was then transferred to a new tube. The cell pellet was washed twice with PBS (pH 7.4) before being homogenized by Pellet Pestle Motor (Kontes, Vineland, N.J.) for 30 sec. All of the cell homogenate and an equal volume of the culture (10 μl) supernatant were added to sheep anti-rabbit IgG coated ELISA plate (Falcon, Oxnard, Calif.) and incubated for 2 h at room temperature. After washing step, HRP-labeled goat anti-rabbit light chain antibody was added, and visualized using o-phenylenediamine. MTS peptide promotes rapid entrance of antibody into cells. The ELISA was designed to capture rabbit immunoglobulin using a sandwich assay. It was observed that the MTS conjugation rapidly promoted monoclonal anti active caspase-3 antibody internalization into the live cells. The translocation of antibodies increased within 1 h and reached a plateau after 18 h. The internalization of naked antibody was delayed (at 3 h) and remained at a lower level when compared to the MTS conjugated-anti-caspase 3 antibody.

The present invention has been described herein with reference to certain examples for purposes of clarity and illustration. It should be appreciated that obvious improvements and modifications of the present invention can be practiced within the scope of the appended claims.

References

The pertinent disclosures of the following references are incorporated herein by reference:

1. Y. Zhao, D. Lou, J. Burkett and H. Kohler, “Enhanced Anti-B-cell Tumor Effects with Anti-CD20 Superantibody,” J Immunotherapy, 25: 57-62, 2002.

2. Ghetie M A, Podar E M, Ilgen A, Gordon B E, Uhr J W, and Vitetta E S, “Homodimerization of tumor-reactive monoclonal antibodies markedly increases their ability to induce growth arrest or apoptosis of tumor cells,” Proc. Natl. Acad. Sci. USA, 94: 7509-7514, 1997.

3. Kaveri S V, Halpern R, Kang C Y, and Kohler H., “Self-binding antibodies (autobodies) form specific complexes in solution,” .J Immunol. 145: 2533-2538, 1990.

4. Kang, C-Y. and Kohler, H., “Immunoglobulin with complementary paratope and idiotope,” J. Exp. Med. 163: 787, 1986.

5. Kohler H, Paul S., “Superantibody activities: new players in innate and adaptive immune responses,” Immunol. Today, 19: 221-7, 1998.

6. Kohler H., “Superantibodies: synergy of innate and acquired immunity,” Appl. Biochem. Biotechnol., 83: 1-9, 2000.

7. U.S. Pat. No. 5,800,991 for “Nucleotide or nucleoside photoaffinity compound modified antibodies, methods for their manufacture and use thereof as diagnostics and therapeutics,” issued to Haley et al., 1998.

8. U.S. Pat. No. 6,238,667 for “Method of affinity cross-linking biologically active immunogenic peptides to antibodies,” issued to Kohler, 2001.

9. U.S. patent Pub. No. 2003/0103984 of U.S. application Ser. No. 09/865,281, filed May 29, 2001 for “Fusion proteins of biologically active peptides and antibodies.”

10. U.S. Pat. No. 6,482,586 for “Hybrid compositions for intracellular targeting,” issued to Arab et al., 2002.

11. U.S. Pat. No. 6,406,693 for “Cancer treatment methods using antibodies to aminophospholipids,” issued to Thorpe et al., 2002.

12. Zhao Y., Brown T., Kohler H., and Müller S., “MTS-Conjugated-Antiactive caspase-3 antibodies Inhibit Actinomycin D-induced apoptosis,” in press, 2003.

13. Lin Y Z, Yao S Y, Veach R A, Torgerson T R, Hawiger J., “Inhibition of nuclear translocation of transcription factor NF-kB by a synthetic peptide containing a cell membrane-permeable motif and nuclear localization sequence,” J. Biol Chem, 270: 14255-14258, 1995.

14. Kang C Y, Brunck T K, Kieber-Emmons T., et al. “Inhibition of self-binding antibodies (autobodies) by a VH-derived peptide,” Science, 240:1034-6, 1988.

15. Kaveri S., Halpern R., Kang C Y., et al., “Antibodies of different specificities are self-binding: implication for antibody diversity,” Mol. Immunol., 2;733-78, 1991.

What is claimed is:

1. A method of treating a patient suffering from a debilitating or life threatening disease comprising administering at least one autophilic antibody to the patient in an amount effective to alleviate symptoms of the disease.

2. The method of claim 1, wherein a characteristic of the disease is malignancy, auto-immune disorder, transplantation rejection, Alzheimer's disease, or other neuro-degenerative condition.

3. The method of claim 1, wherein the autophilic antibody is administered in one or more dose amounts substantially identical to that practicable for naked antibodies.

4. The method of claim 3, wherein an initial dose is about 250 mg per day and a later dose is about 100 mg per week.

5. A method of potentiating apoptosis of selected cells in a patient comprising administering to the patient a first autophilic antibody-peptide conjugate and a second antibody directed to the autophilic peptide itself.

6. A method of producing an autophilic antibody by chemical or genetic engineering techniques, wherein the autophilic antibody contains a T15 autophilic peptide sequence (ASRNKANDYTTDYSASVKGRFIVSR) that attaches via a tryptophan photoactivation crosslinking to the immunoglobulin component of the antibody.

7. The method of claim 6, wherein the T15 peptide of the autophilic antibody is crosslinked to a nucleotide affinity site of the immunoglobulin.

8. The method of claim 6, wherein the T15 peptide is crosslinked to a carbohydrate site of the Fc portion of the immunoglobulin.

9. The method of claim 6, wherein the T15 peptide is conjugated to an amino or sulfhydryl group of the immunoglobulin.

10. The method of claim 6, wherein the autophilic antibody is expressed as a fusion protein containing the T15 autophilic sequence.

11. A method of formulating an autophilic antibody composition so as to reduce or mitigate dimerization in solution comprising addition of salt concentrations of 0.5M or more, low levels of SDS, various detergents especially those of an anionic nature, or modifications of antibody to decrease its isoelectric point as with succinyl anhydride.

12. A method of expressing an increased degree of apoptosis in an in vitro assay of an antibody/antigen system comprising employing an autophilic conjugate.

13. A method of identifying an autophilic antibody candidate for use in humans comprising administering the autophilic antibody to SCID or nude mice having human tumor xenografts.

14. A method of determining a peptide sequence for enhanced noncovalent autophilic coupling between antibody molecules: comprising providing a plurality of synthetic peptides each having one or more conservative substitutions at amino acid positions of a template peptide; and comparing self-binding properties of such synthetic peptides relative to those of the template peptide.

15. The method of claim 14, wherein the template peptide is the T15 peptide.

16. A method of producing an autophilic antibody by chemical or genetic engineering techniques, wherein the autophilic antibody contains a modified T15 autophilic peptide sequence that further potentiates the ability of the modified autobody to crosslink once bound to a target antigen relative to the unmodified autobody.

17. A conjugate antibody wherein two or more bioactive peptides are conjugated to different sites of an antibody to potentiate therapeutic efficacy.
